(19) United States
(12) Patent Application Publication
(10) Pub. No.: US 2013/0210881 A1
(43) Pub. Date: Aug. 15, 2013

(54) ANTIMICROBIAL COMPOSITION

(71) Applicants: Yugen Zhang, Singapore (SG); Lihong Liu, Singapore (SG)

(72) Inventors: Yugen Zhang, Singapore (SG); Lihong Liu, Singapore (SG)

(73) Assignee: Agency for Science ,Technology and Research, Singapore (SG)

(21) Appl. No.: 13/879,615

(22) PCT Filed: Oct. 13, 2011
(86) PCT No.: PCT/SG2011/000357

§ 371 Date: Apr. 15, 2013

(30) Foreign Application Priority Data
Oct. 15, 2010 (SG) 201007600-8

(51) Int. Cl. C07D 403/14

(52) U.S. Cl. CPC C07D 403/14
USPC 514/397; 548/313.7

(57) ABSTRACT

The present disclosure relates to an antimicrobial composition comprising at least one polymer or oligomer, the polymer and oligomer being comprised of repeating units of hydrophilic heterocyclic amine monomers that are coupled by hydrophobic linkers selected to confer the antimicrobial activity to the composition, methods of producing the same and uses of the antimicrobial composition.

### TECHNICAL FIELD

The present invention relates to antimicrobial compositions and the use of such compositions in biocidal, antimicrobial and antifungal applications.

### BACKGROUND

Antibiotics were first isolated and used to treat bacterial infections in 1939. Over the last 70 years, antibiotics and other anti-microbial compounds have been at the forefront of the fight against infectious diseases. However, long term exposure to such compounds has allowed pathogens to evolve and develop resistance to the different drugs and their mechanisms of action. Drug resistant pathogens have been found in ever increasing strains and pathogens that are resistant to multiple drug types have also been discovered in the medical community. In the present environment, clinicians and researchers face increasing difficulties in developing suitable antibiotics that utilise a novel mechanism of action against pathogens un-hampered by resistance developed from other antibiotics, act effectively against the pathogens within a short exposure period and are non-toxic to mammalian cells in a concentration significantly higher than that which affects the pathogens.

Host defense antimicrobial peptides (“AMPs”) are innate components of an organism's immune system that are potent and broad spectrum antimicrobial compounds. AMPs have provided a new direction in the development of antibiotics as they break away from the typical inhibitory mechanism of traditional antibiotics as the postulated mechanism for their activity is based on diffusion into and disruption of the cytoplasmic membrane, leading to bacteria death. This mechanism targets a generic characteristic common to the membranes of many pathogenic species and it is thought that resistance to such mechanisms may be slower to develop. This mechanism is thought to be possible, due to the residues in the AMPs adopting highly amphiphilic conformations in which the cationic hydrophilic and hydrophobic groups segregate into distinct sections or regions in the molecular structure, facilitating the interactions between the AMPs and the bacterial cytoplasmic membrane. However, isolation from natural sources and chemical synthesis of the AMPs has not proven to be cost-effective.

Hence, there has been considerable interest in the development of synthetic analogues or similar polymers or oligomers. Such synthetic polymers or oligomers should capture the characteristics of the AMP that have been surmised to contribute to their antimicrobial activities, in particular, the cationic hydrophilic groups and hydrophobic moieties. These synthetic polymers or oligomers should also preferably be relatively inexpensive, easy to synthesize, possess a wide range of molecular weights and should possess characteristics such as being non-toxic to mammalian cells, yet active against a wide spectrum of pathogens with short contact duration.

A recent study has managed to produce quaternary ammonium or phosphonium functionalized polymers with excellent biocidal activities however, these polymers have been shown to be highly toxic to mammalian cells. Hence, they can only be used as disinfectants, biocidal coatings or filters. Another study managed to synthesize polymers with non-hemolytic properties. However, these polymers were designed using arylamide, phenylene-ethynylene, acrylate and other hydrocarbon-based polymers and materials, depending on a rigid structure substituted with cationic and hydrophobic moieties to achieve the necessary facial segregation of cationic and hydrophilic groups to achieve the amphiphilic and amphipathic characteristics of AMPs.

Accordingly, there is a need to provide an antimicrobial composition, which overcomes, or at least ameliorates one of the disadvantages mentioned above.

### SUMMARY

In a first aspect, there is provided an antimicrobial composition comprising at least one polymer or oligomer, said polymer and oligomer being comprised of repeating units of hydrophilic heterocyclic amine monomers that are coupled by hydrophobic linkers selected to confer the antimicrobial activity to the composition.

Advantageously, the present invention provides an alternative to existing AMPs, which are typically produced naturally in-vivo by immune systems of living organisms but are hard to isolate and replicate in an ex-vivo environment. Advantageously, the present invention provides easy-to-produce, pharmacologically active, cost-effective and pharmaceutically safe antimicrobial compositions which are capable of simulating the antimicrobial properties of naturally produced AMPs.

In one embodiment, the disclosed antimicrobial composition is capable of inhibiting the growth of or killing pathogenic microorganisms by disrupting the structural integrity of the lipid bilayer, i.e., the main component of the cytoplasmic membrane. In this regard, it is postulated that the amphiphilic nature of the polymers or oligomers allows them to assume a cationic, amphipathic conformation. Specifically, the polymer or oligomer is capable of assuming an overall folded configuration, wherein its hydrophilic sections and hydrophobic sections segregate into distinct, facially opposing regions. This facially amphiphilic topology in turn facilitates the polymer's insertion into an anionic, hydrophobic cell membrane, thereby disrupting the structural integrity of the cell membrane, which eventually leads to cell death.

Accordingly, the hydrophobic linkers must be appropriately selected to provide sufficient structural rigidity but at the same time, confer enough flexibility to the polymeric/oligomer structure such that a facially amphiphilic topology can be formed.

Further advantageously, it has been surprisingly found that the disclosed antimicrobial composition, contrary to conventional synthetic AMPs, exhibits a relatively low propensity for hemolysis, i.e., the killing red blood cells (RBCs). Therefore, the disclosed composition is capable of selectively killing pathogenic microorganisms whilst exerting little or no adverse toxic effects in an organism administered with the composition.

In a second aspect, there is provided a microbial cream comprising the antimicrobial composition as defined above along with one or more pharmaceutically acceptable excipients suitable for topical administration.

In a third aspect, there is provided a microbial composition as defined above for use as in therapy.

In a fourth aspect, there is provided the use of the composition as defined above, for the preparation of a medicament for treating bacterial infections.

In a fifth aspect, there is provided a method of producing an amphiphilic polymer or oligomer, the method comprising a step of reacting aryl-substituted heterocyclic amine monomers units with at least one of a heterocyclic amine substituted with one or more haloalkyl arenes, haloalkyl arenes, and a di-halogenated aliphatic olefin, in the presence of an organic solvent, said aryl-substituted heterocyclic amine monomers units having at least two heterocyclic amine groups linked by an aryl group.

### Definitions

The following words and terms used herein shall have the meaning indicated:

The term “amphiphilic polymer” as used in the context of the present specification, refers to a polymer having discrete hydrophilic and hydrophobic regions, wherein the discrete hydrophilic and hydrophobic regions are arranged in a facially amphiphilic conformation, i.e., the hydrophilic and hydrophobic regions are opposite facing relative to one another.

The terms “antimicrobial”, “biocidal” or “antifungal”, as used herein, refers interchangeably to a form of bioactivity exhibited by compounds, which inhibits or destroys the growth of microorganisms. Such bioactivity may include killing of the microorganisms or simply stagnating the growth of such microorganisms.

The term “microorganism” as used herein, refers broadly to both eukaryotic and prokaryotic organisms possessing a cell membrane, including but not limited to, bacteria, yeasts, fungi, plasmids, algae and protozoa.

The term “heterocyclic”, as used in the context of the present specification, refers to a cyclic compound having at least one ring structure, wherein the ring structure is composed of at least two different elements.

The term “heterocyclic amine” as used in the context of the present specification, refers to a cyclic compound having at least one ring structure, wherein the ring structure contains at least one nitrogen atom and at least one other atom that is not nitrogen, said nitrogen atom forming a primary, secondary or tertiary amine functional group on said cyclic compound.

The term “haloalkyl arenes” as used in the context of the present specification refers to monocyclic or polycyclic aromatic hydrocarbons which are substituted by one or more aliphatic C₁₋₁₀ alkyl groups, said alkyl groups being substituted by one or more halogens.

The word “substantially” does not exclude “completely” e.g. a composition which is “substantially free” from Y may be completely free from Y. Where necessary, the word “substantially” may be omitted from the definition of the invention.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### DISCLOSURE OF OPTIONAL EMBODIMENTS

Exemplary, non-limiting embodiments of an amphiphilic polymer according to the first aspect will now be disclosed.

In the disclosed antimicrobial composition, the heterocyclic amines monomer units may be selected from heterocyclic amines having a 4-membered ring, a 5-membered ring or a 6-membered ring.

The heterocyclic amines monomer units may be selected from the group consisting of: azetidine, dihydroazete, pyrolidine, imidazolidine, triazolidine, tetrazolidine, pentazolidine, pyrrole, imidazole, triazole, pyridine, piperidine, diazinane, triazinane, pyrimidine, triazine and combinations thereof.

Advantageously, the nitrogen atom residing on the heterocyclic ring is capable of forming bonds with hydrophobic linker groups to form an extended polymeric or oligomeric structure. Further advantageously, as the lone pair of unbound electrons become “consumed” in the formation of covalent bonds, the nitrogen atom may become electron-deficient (or an “electrophile”) and hence assume a cationic property. This cationic property renders the heterocyclic amine monomer unit polar and hydrophilic. The polarity and hydrophilicity in turn help the polymer or oligomer to generate electrostatic interactions with a typically anionic cell membrane, facilitating insertion into and subsequent disruption of the cell membrane. Also advantageously, the cationic nature of the heterocyclic amine monomer unit may assist the amphiphilic polymer/oligomer to “self-assemble” into a folded, facially amphiphilic conformation, having opposing faced hydrophilic and hydrophobic regions.

In one embodiment, the heterocyclic amine monomer unit is an imidazole unit. In another embodiment, the heterocyclic amine monomer unit is a triazole unit.

The hydrophobic linker may be selected from an optionally substituted aryl group and an aliphatic olefin. Advantageously, it has been found that linker groups selected from a substituted aryl group and an aliphatic, olefin group are most suitable for achieving the required facially amphiphilic topology. Without wishing to be bound by theory, it is postulated that the large, substituted aryl group provides a relatively high degree of hydrophobicity to the amphiphilic polymer. Additionally, the planar sp² π bonding of the “C═C” double bond in the aliphatic olefin strengthens the rigidity of the hydrophobic region. This in turns facilitates the arrangement of the hydrophobic and hydrophilic groups into distinct, facially opposed regions.

The antimicrobial composition may comprise a polymer or an oligomer, having repeating units of general formula (I),

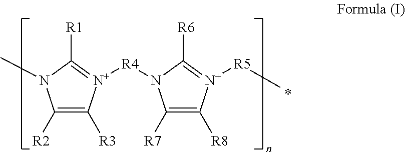

wherein:

R4 and R5 are independently selected from the group consisting of optionally substituted aryl and an aliphatic olefin and;

R1, R2, R3, R6, R7, and R8 are independently selected from hydrogen, alkyl, alkenyl, aryl, halogen and amines; and

n is an integer of at least two.

In another embodiment, the integer n may be in a range of from 2 to 50, from 2 to 40, from 2 to 30, from 2 to 10, from 3 to 50, from 3 to 40, from 3 to 30, from 3 to 10, from 4 to 50, from 4 to 40, from 4 to 30, from 4 to 10, from 5 to 50, from 5 to 40, from 5 to 30, from 5 to 10, from 6 to 50, from 6 to 40, from 6 to 30, from 6 to 20, or from 6 to 10. In one embodiment, n is from at least 6 to 8. In one embodiment, the above composition comprises a polymer, wherein n is from 8 to 50. In another embodiment, the above composition comprises an oligomer, wherein n is 4 to 10.

In the disclosed antimicrobial composition, the hydrophobic linker groups R4 and R5 may be independently selected from the group consisting of: xylene, aliphatic C₂₋₆ alkylene, phenylbenzene, substituted phenylbenzene, and combinations thereof. In particular, R4 and R5 may be independently selected from the group consisting of: ortho-xylene, para-xylene, meta-xylene, pyridine, butylene, substituted bi-phenyl, propene, ethene, and combinations thereof. In one embodiment, the bi-phenyl linker is 1-methyl-4-(4-methylphenyl)benzene.

Advantageously, it has been found that amphiphilic polymers/oligomers comprising the above defined hydrophobic linkers exhibit strong antimicrobial activity, whilst at the same time, possess limited or negligible hemolytic effects.

In one embodiment, limited or negligible hemolytic effects means that the HC₅₀ concentration (which is the concentration of the compound required to kill 50% of given concentration of red blood cells) of the disclosed polymer is, at least 10 times higher than its minimum inhibitory concentration (MIC) with respect to a defined microorganism. In another embodiment, the HC₅₀ concentration of the polymer is at least 15 times that its MIC value. In yet another embodiment, the HC50 concentration of the polymer is at least 25 times that of its MIC value. In general, the higher the multiple of HC50 relative to its MIC value, the safer the polymer is for administration to a living organism.

In a preferred embodiment of the disclosed polymer/oligomer, R4 is ortho-xylene and R5 is butylene. Advantageously, this particular combination of imidazole monomers with ortho-xylene and butylene hydrophobic linkers has been found to possess exceptional antimicrobial properties. As will be further discussed in the Examples section below, an antimicrobial composition comprising the above disclosed combination of imidazole monomers and linkers exhibits a MIC of 20 μg/ml against the microbe E. Coli, a MIC of 7.8 μg/ml against B. subtilis and a MIC of 35 μg/ml against C. albicans. More importantly, this particular embodiment exhibits negligible hemolytic properties with its HC₅₀ value far exceeding 500 μg/ml.

In one embodiment, the substituent groups R1, R2, R3, R6, R7, and R8 are each hydrogen.

In the antimicrobial composition, the polymer may be provided as a halide salt. The halide may be formed from a halogen selected from the group consisting of fluorine, bromine, chlorine or iodine. In one embodiment, the halogen is bromide. In another embodiment, the halogen is chloride.

In the antimicrobial composition according to the first aspect, the oligomer may comprise at least four imidazolium units, each imidazolium unit being coupled to an adjacent imidazolium unit via a hydrophobic linker molecule A and said imidazolium unit having the general formula (II):

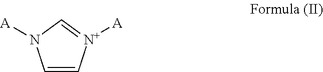

The linker molecule A may be independently selected from an optionally substituted aryl and an aliphatic olefin.

In one embodiment, the oligomer has at least six imidazolium units. Advantageously, it has been found that antimicrobial activity of the oligomer becomes more potent when the oligomer comprises at least six imidazolium units in the oligomer backbone. Without wishing to be bound by theory, it is postulated that if the oligomer chain is too short, the degree of interaction between the oligomer molecule and the cell membrane's lipid bilayer will be relatively weak. On the other hand, an oligomer having too long a chain may be too hydrophobic and lack solubility, which may lead to aggregation and also a higher rate of hemolysis.

In one embodiment, the linker group A may be selected from the group consisting of:

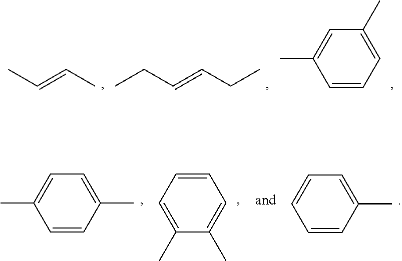

In one embodiment, the oligomer may comprise four imidazolium units, each imidazole coupled to another imidazole group via a linker group A, which is independently selected from the compounds provided above.

The terminal ends of the disclosed oligomer may be capped by an aryl group. In one embodiment, the oligomer is capped at both ends by a phenyl group.

In one embodiment, the disclosed oligomer may be selected from the group consisting of:

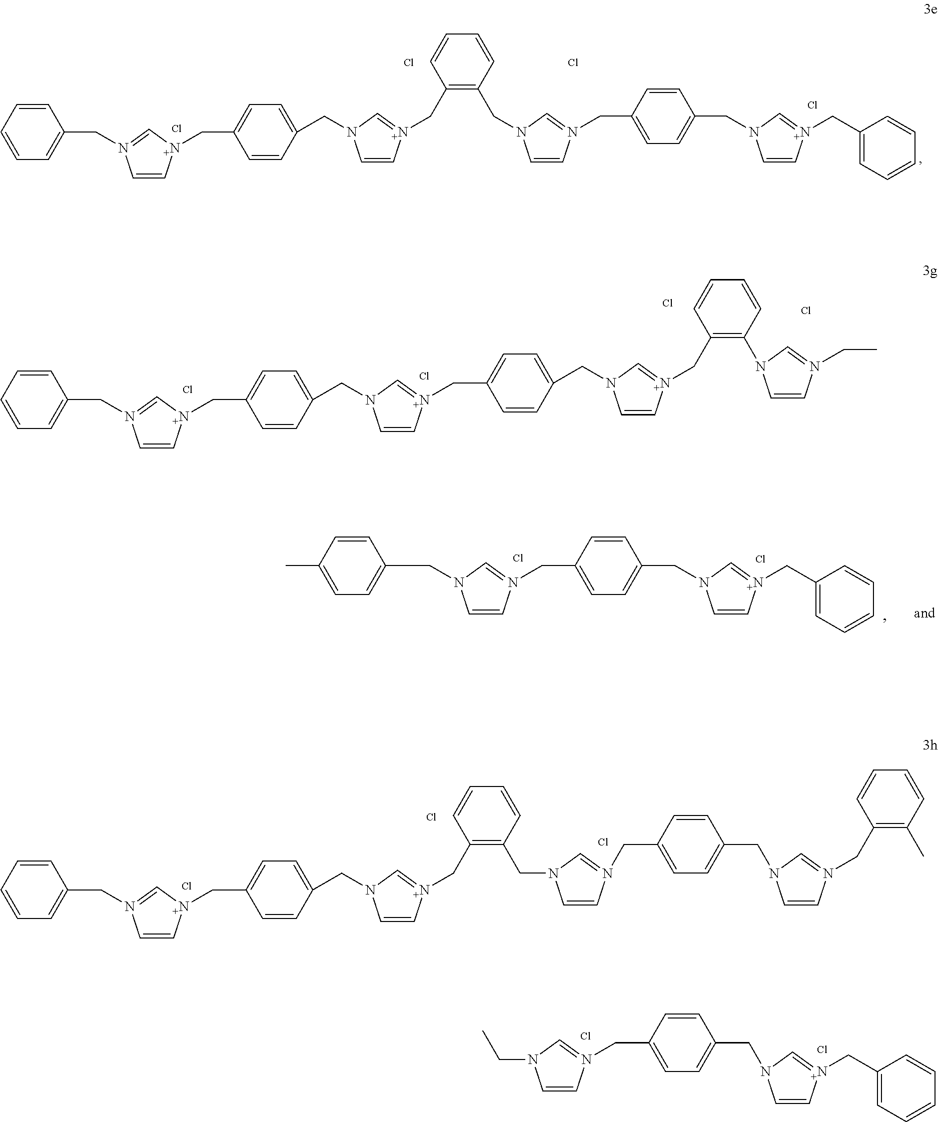

The oligomer may be provided as an oligomeric halide salt. In one embodiment, the halide of the oligomeric halide salt is selected from fluoride, bromide, chloride or iodide.

Exemplary, non-limiting embodiments of microbial cream according to the second aspect will now be disclosed.

The microbial cream may comprise the antimicrobial composition as defined above together with one or more pharmaceutically acceptable excipients suitable for topical administration. The antimicrobial composition may comprise an amphiphilic polymer, or an amphiphilic oligomer or a mixture thereof. Suitable pharmaceutical excipients for use in topical applications are within the expertise of a person skilled in the art.

In one embodiment, suitable pharmacologically acceptable excipients may include hydrocarbon bases such as white petrolatum, anhydrous absorption bases, hydrophilic petrolatum and anhydrous lanolin and water-in-oil emulsion bases.

In another embodiment, the pharmaceutically acceptable excipients may include excipients which are substantially non-occlusive, and which are water-soluble, such as oil-in-water emulsion bases and water-soluble bases such as polyethylene glycol-based excipients and aqueous solutions comprising methylcellulose, hydroxyethyl cellulose, and hydroxypropyl methylcellulose.

Exemplary, non-limiting embodiments of the use according to the fourth aspect will now be disclosed. The disclosed antimicrobial composition may be used in the preparation of a medicament for treating bacterial infections. The bacterial infections may be selected from infections caused by gram-positive or gram-negative bacteria. Particularly, the bacteria causing the infection may be selected from the group consisting of: Bacillus subtilis, Vancomycin-resistant enterococcus, methicillin-resistant Staphylococcus aureus (MRSA), Escherichia coli, Klebsiella pneumoniae, Candida albicans, and Cryptococcus neoformans. In one embodiment, the bacterial infections to be treated may be caused by Escherichia coli, Bacillus subtilis and Candida albicans.

The medicament may be prepared in a form that is suitable for administration intravenously, topically, nasally, orally, sublingually, or subcutaneously.

Exemplary, non-limiting embodiments of the method according to the fifth aspect will now be disclosed.

In the disclosed method, the aryl-substituted heterocyclic amine monomer unit may be formed from an earlier pre-reaction step between imidazole and one or more di-halogenated xylenes to provide the aryl-substituted heterocycle amine monomer unit. This pre-reaction step may be undertaken at room temperature in the presence of an organic solvent and optionally a metal catalyst. In one embodiment, this pre-reaction step is undertaken in the presence of N,N′-dimethylformamide (DMF) and with sodium hydride (NaH). Other suitable organic solvents such as, but not limited to, tetrahydrofuran and acetonitrile, are also within the scope of the present disclosure. In one embodiment, DMF is the preferred solvent.

In one embodiment, the aryl-substituted heterocycle amine monomer unit formed from the pre-reaction step comprises at least two imidazole units linked to each other via xylene linker, selected from p-xylene, o-xylene and m-xylene. In another embodiment, the formed aryl-substituted heterocyclic amine monomer unit comprises at least two imidazole units linked to each other via a biphenyl group.

In one embodiment, in order to prepare the polymer according to the present invention, the formed aryl-substituted heterocyclic amine monomer unit may be subsequently reacted with a di-halogenated aliphatic olefin selected from the group consisting of: di-bromobutylene, di-chlorobutylene, di-chloropropene, di-bromopropene, di-chloroethylene, di-bromoethylene, and mixtures thereof. This polymerization step may be undertaken in the presence of an organic solvent such as DMF, wherein the reaction mixture is stirred for about 5 hours at about 100° C.

In another embodiment for forming the polymer according to the present invention, the aryl substituted heterocyclic amine monomer unit may be reacted with one or more haloalkyl arenes selected from the group consisting of: dibromo-m-xylene, dibromo-p-xylene, dibromo-o-xylene, dichloro-m-xylene, dichloro-p-xylene, dichloro-o-xylene, 4,4′-bis(chloromethyl)-1,1′-biphenyl and mixtures thereof. This polymerization step may be undertaken in the presence of an organic solvent such as DMF, wherein the reaction mixture is stirred for 5 hours at about 100° C.

In another embodiment of the method disclosed herein, an oligomer may be formed by reacting the earlier formed aryl-substituted heterocyclic amine monomer unit with one or more of another heterocyclic amine that has been substituted with one or more haloalkyl arenes.

In one embodiment, the haloalkyl arene substituted heterocyclic amine may comprise from one to four imidazole units, each imidazole unit being linked to another imidazole unit via a haloalkyl arene linker, wherein the haloalkyl arene is as defined above. In another embodiment, the haloalkyl arene substituted heterocyclic amine may be capped at its terminal ends by an haloalkyl arene group.

In one embodiment, the haloalkyl arenes substituted heterocyclic amine is selected from the group consisting of:

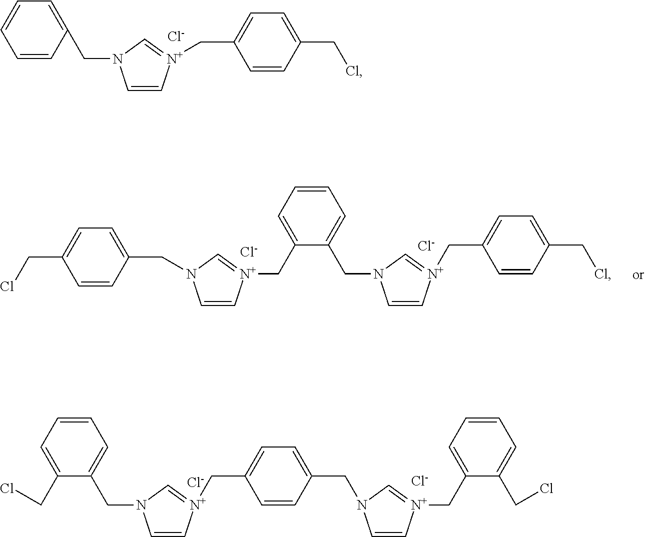

This step of forming the oligomers may be undertaken in the presence of an organic solvent such as DMF, wherein the reaction mixture is stirred for 5 hours at about 90° C.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 shows the toroidal model of antimicrobial peptide-induced cell killing.

FIG. 2(A) shows an exemplary amphiphilic polymer structure with a rigid imidazole backbone.

FIG. 2(B) shows the folding and “self-assembling” action of an amphiphilic polymer into an amphipathic conformation when exposed to a cell membrane surface.

FIG. 2(C) shows an exemplary polymer/oligomer according to the present invention assuming a substantially folded configuration comprising a facially amphiphilic topology.

FIG. 3 is a graph comparing the hemolytic activity of the various polymers and oligomers.

FIG. 4(a) shows B. subtilis units of colony formation after being treated with sample 2f at various time intervals.

FIG. 4(b) shows E. coli units of colony formation after being treated with sample 2f at various time intervals.

FIG. 5 shows combined confocal fluorescence images of E. coli before and after addition of sample 2f.

FIG. 6(a) shows a photograph of rat red blood cells without addition of polymer sample (control).

FIG. 6(b) shows a photograph of rat red blood cells in a solution with 31.25 parts per million (ppm) of sample 2f.

FIG. 6(c) shows a photograph of rat red blood cells in a solution with 62.5 parts per million (ppm) of sample 2f.

FIG. 6(d) shows a photograph of rat red blood cells in a solution with 125 parts per million (ppm) of sample 2f.

FIG. 6(e) shows a photograph of rat red blood cells in a solution with 250 parts per million (ppm) of sample 2f.

FIG. 6(f) shows a photograph of rat red blood cells in a solution with 500 parts per million (ppm) of sample 2f.

FIG. 7(a) shows a photograph of rat red blood cells without addition of any polymer sample (control)

FIG. 7(b) shows a photograph of rat red blood cells in a solution with 7.8 parts per million (ppm) of polymer sample 2l.

FIG. 7(c) shows a photograph of rat red blood cells in a solution with 62.5 parts per million (ppm) of polymer sample 2l.

FIG. 7(d) shows a photograph of rat red blood cells in a solution with 125 parts per million (ppm) of polymer sample 2l.

FIG. 7(e) shows a photograph of rat red blood cells in a solution with 250 parts per million (ppm) of polymer sample 2l.

FIG. 7(f) shows a photograph of rat red blood cells in a solution with 500 parts per million (ppm) of polymer sample 2l.

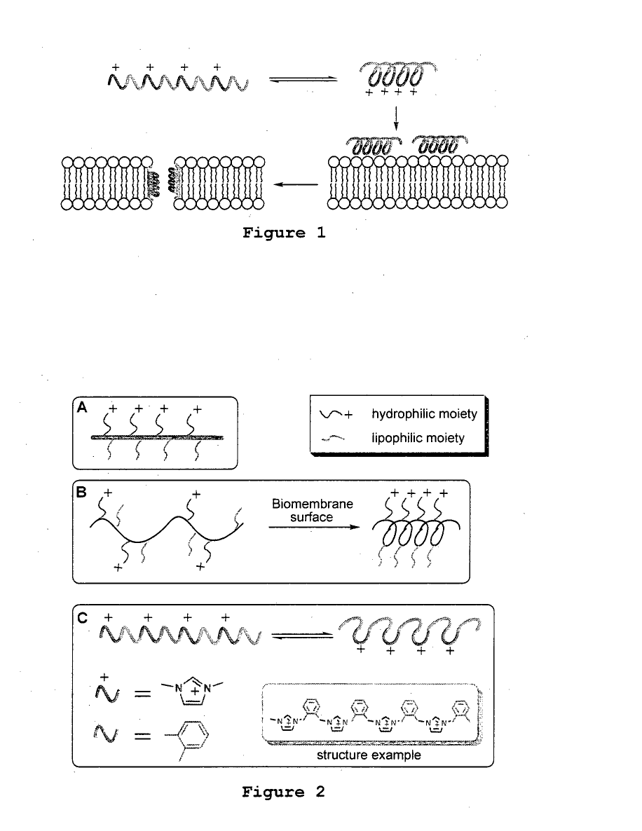

### EXAMPLES

Non-limiting examples of the invention will be further described in greater detail by reference to specific Examples, which should not be construed as in any way limiting the scope of the invention.

All solvents and chemicals were used as obtained from commercial suppliers, unless otherwise indicated. Triton-X was obtained from Sigma-Aldrich, United States of America. Live/Dead Baclight bacterial viability kits for staining bacteria were bought from Invitrogen Technologies, Singapore.

### Example 1

Synthesis of Monomer Intermediates 1a-1e

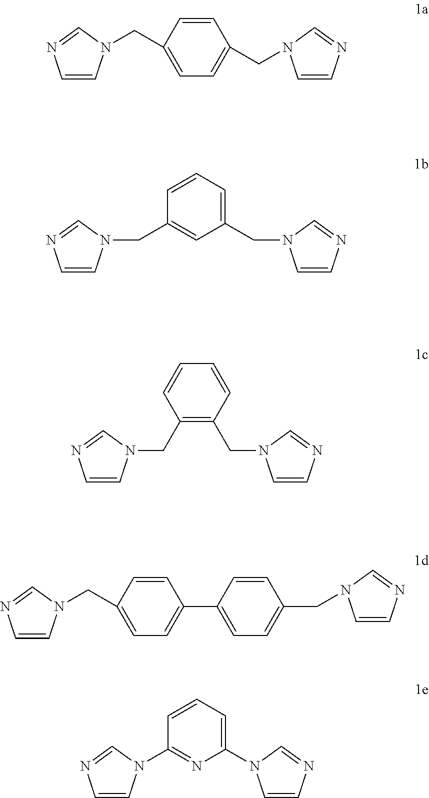 [SMILES: c1cn(Cc2ccc(Cn3ccnc3)cc2)cn1 ; c1cc(Cn2ccnc2)cc(Cn2ccnc2)c1 ; c1ccc(Cn2ccnc2)c(Cn2ccnc2)c1 ; c1cn(Cc2ccc(-c3ccc(Cn4ccnc4)cc3)cc2)cn1 ; c1cc(-n2ccnc2)nc(-n2ccnc2)c1]

Sodium Hydride (NaH) (60% in oil, 440 mg, 11 mmol) was added to a N,N′-dimethylformamide (DMF) solution of imidazole (680 mg, 10 mmol), and the resulting suspension was stirred at room temperature for two hours. Subsequently, a,a′-dichloro-p-xylene (5 mmol) was added to the residue and the solution was stirred at room temperature for another four hours.

The product was extracted from the reaction mixture using dichloromethane (DCM) and the resultant extract was dried by using a vacuum to remove the solvent to obtain sample 1a in quantitative yield.

Using the above protocol, three other monomer intermediates were prepared, by replacing the reactant a,a′-dichloro-p-xylene with the following reactants for each of the monomer intermediates

Sample 1b: a,a′-dibromo-m-xylene

Sample 1c: a,a′-dibromo-o-xylene

Sample 1d: 4,4′-bis(chloromethyl)-1,1′-biphenyl

Further analysis was done on samples 1a, 1b and 1d using Nuclear Magnetic Resonance (NMR) and Gas Chromatography-Mass Spectroscopy (GC-MS) to yield the following results.

### Sample 1a:

¹H NMR (CDCL₃): δ 7.55 (s, 2H), 7.13 (s, 4H), 7.10 (s, 2H), 6.89 (s, 2H), 5.12 (s, 4H).

MS (GC-MS): m/z 238 (M⁺).

### Sample 1b:

¹H NMR (MeOD-d4): δ 7.69 (s, 2H), 7.33 (t, 1H), 7.17 (d, 2H), 7.12 (s, 1H), 7.04 (s, 2H), 6.94 (s, 2H), 5.18 (s, 4H).

MS (GC-MS): m/z 238 (M⁺).

### Sample 1d:

¹H NMR (MeOD-d4): δ 7.78 (s, 2H), 7.61 (d, 4H), 7.33 (d, 4H), 7.14 (s, 2H), 7.00 (s, 2H), 5.27 (s, 4H).

MS (GC-MS): m/z 314 (M⁺).

2,6-dibromopridine, imidazole and Na₂CO₃ were mixed in a reactor using a 1:2.5:2.5 mole ratio. The reactor was then closed and heated to 130° C. for 16 hrs. The reaction mixture was subsequently separated using column chromatography to obtain the product, sample 1e.

### Example 2

### Synthesis of Main Chain Polymer Imidazolium (MCPIM)

a,a′-dichloro-p-xylene (5 mmol) was added to the reaction mixture of sample 1a (5 mmol) and the solution was stirred at 100° C. for 5 h. After a white solid product had precipitated in the reaction flask, the suspension was filtered, washed with DMF followed by DCM, and dried under vacuum to produce sample 2a.

Using the above protocol, 19 other MCPIM materials were prepared by the monomer intermediates 1a-e with different linker molecules in DMF according to Table 1 below.

TABLE 1

| Sample | Monomer intermediate | Linker molecule |
|---|---|---|
| 2a | 1e | a,a′-dichloro-p-xylene |
| 2b | 1e | a,a′-dichloro-o-xylene |
| 2c | 1e | dibromomethane |
| 2d | 1e | 1,2-dibromoethene |
| 2e | 1a | terephthaloyl chloride |
| 2f | 1a | a,a′-dichloro-p-xylene |
| 2g | 1a | a,a′-dichloro-m-xylene |
| 2h | 1a | a,a′-dichloro-o-xylene |
| 2i | 1a | 1,4-dibromobutylene |
| 2j | 1c | a,a′-dichloro-o-xylene |
| 2k | 1c | a,a′-dichloro-m-xylene |
| 2l | 1c | 1,4-dibromobutylene |
| 2m | 1d | a,a′-dichloro-p-xylene |
| 2n | 1d | 1,4-dibromobutylene |
| 2o | 1a | dibromomethane |
| 2p | 1a | 1,2-dibromoethene |
| 2q | 1a | 1,3-dibromopropene |
| 2r | 1c | 1,2-dibromoethene |
| 2s | 1c | dibromomethane |
| 2s | 1d | 1,2-dibromoethene |

### Example 3

### Synthesis of Main-Chain Oligomer Imidazolium

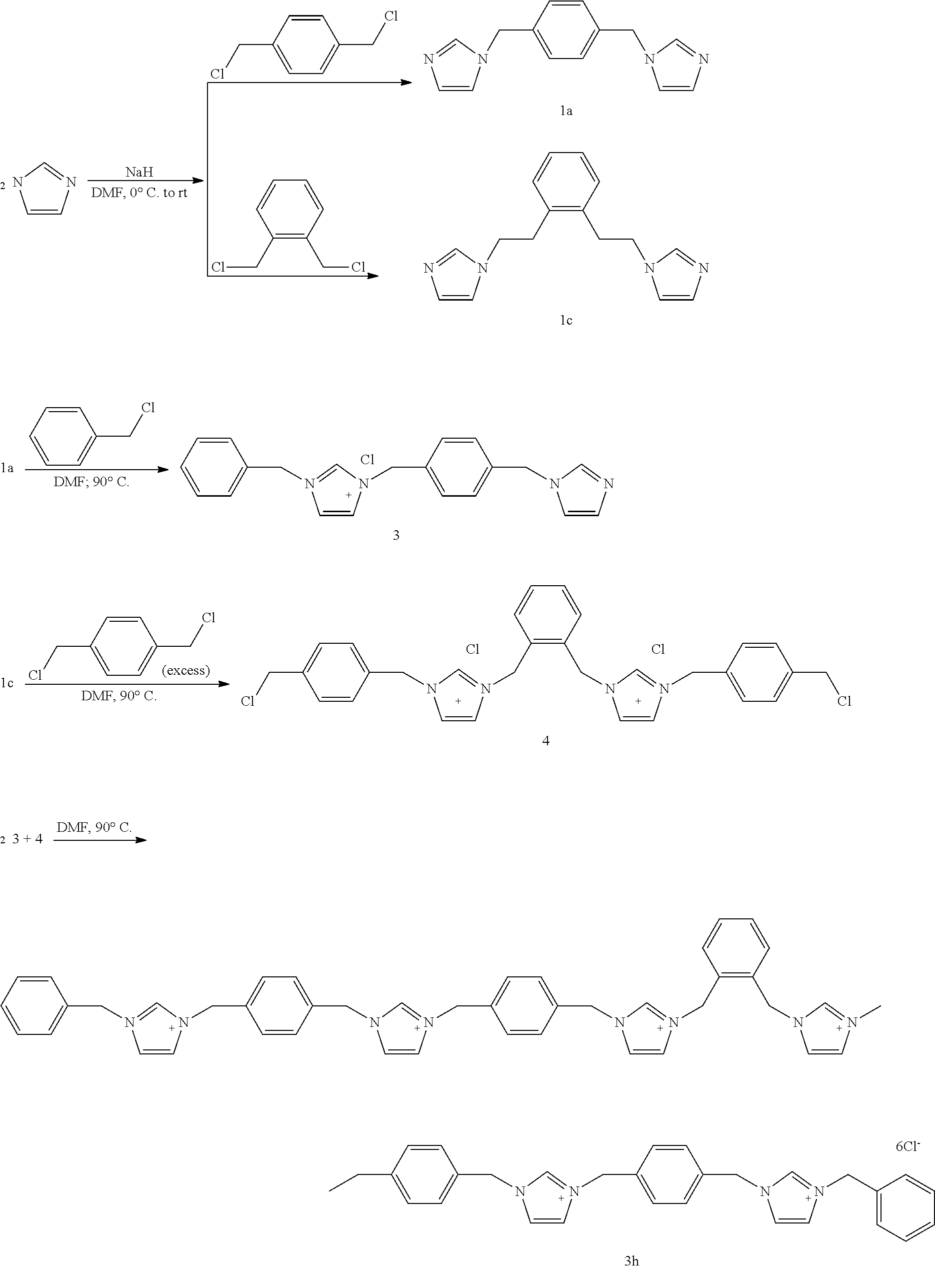

An exemplary method for producing an oligomer according to the present invention is provided in the reaction scheme above.

Firstly, intermediate samples 1a and 1c were synthesized as described above.

For the synthesis of compound 3, benzyl chloride (252 mg, 2 mmol) was dissolved using a DMF solvent base and dropped into the reaction mixture of sample 1a (714 mg, 3 mmol). The resultant mixture was stirred at 90° C. for 8 hours and the solvent was removed under vacuum. Flash column chromatography was then used to obtain the purified compound 3. ¹H NMR (CDCl₃): δ 10.70 (s, 1H), 7.53 (m, 3H), 7.45 (m, 2H), 7.39 (m, 1H), 7.35 (m, 3H), 7.17 (m, 1H), 7.13 (d, 2H), 7.03 (s, 1H), 6.89 (s, 1H), 5.59 (s, 2H), 5.52 (s, 2H), 5.11 (s, 2H).

For the synthesis of compound 4, sample 1c (238 mg, 1 mmol) was added to a N,N′-dimethylformamide (DMF) solution of a,a′-dichloro-p-xylene (5 mmol) and the resultant mixture was stirred at 90° C. for 8 hours. The reaction mixture was then cooled, filtered to remove insoluble impurities, and the solvent was removed under vacuum. Crystallization was subsequently used to obtain a purified compound 4. ¹H NMR (CD₃OD): δ 7.35-7.70 (m, 18H), 5.68 (s, 4H), 5.52 (s, 4H), 5.43 (s, 4H).

For the synthesis of sample 3h, compound 3 (364 mg, 1 mmol) was added to a N,N′-dimethylformamide (DMF) solution of compound 4 (294, 0.5 mmol) and the resultant mixture was stirred at 90° C. for 8 hours. The reaction mixture was decanted to leave the solid precipitate, which was subsequently washed by DMF and re-crystallized from methanol solution to give sample 3h in 90% yield. ¹H NMR (CD₃OD): δ 7.35-7.70 (m, 48H), 5.45 (s, 4H), 5.47 (s, 4H), 5.49 (s, 8H), 5.51 (s, 4H), 5.72 (s, 4H). MALDI-TOF-MS: m/z 185 (M⁶⁺+1)

The above protocol was used to synthesis 7 other samples, varying the reactants and intermediates according to the equations provided below.

### Sample 3a:

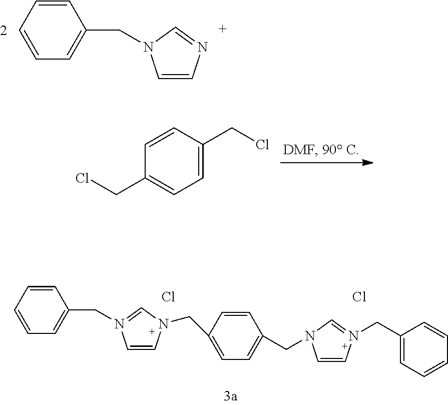

### Sample 3b:

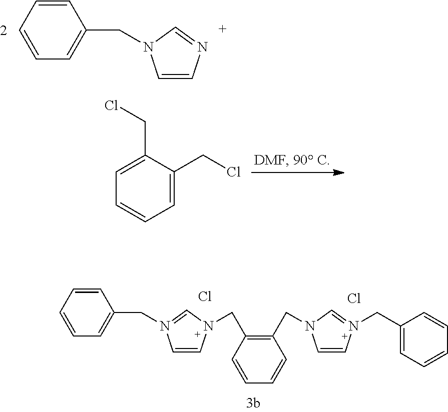

### Sample 3c:

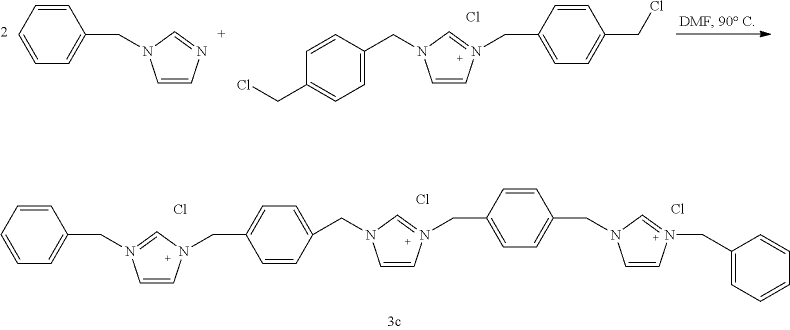

### Sample 3d:

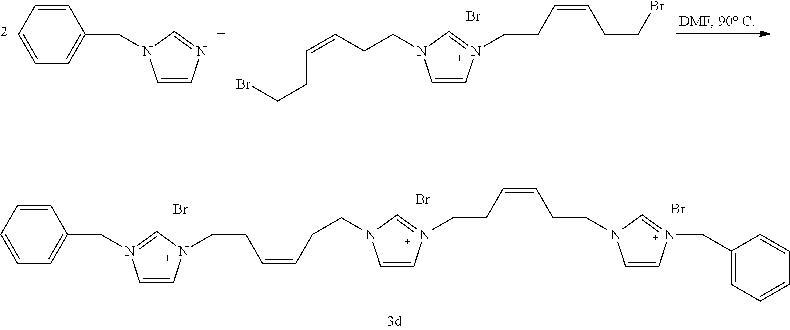

### Sample 3e:

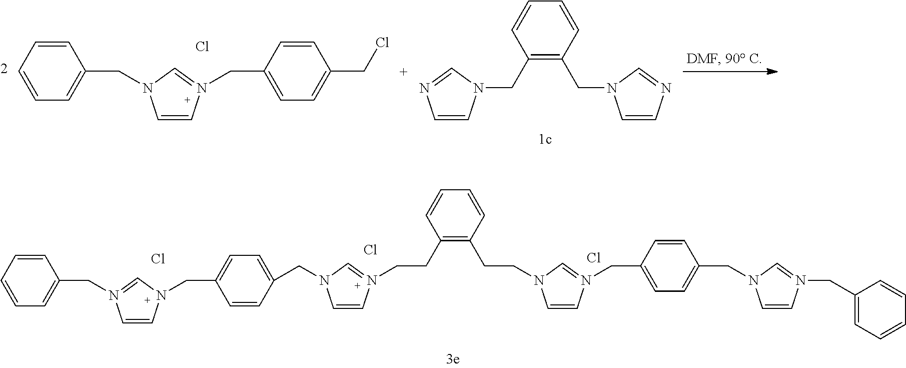

### Sample 3f:

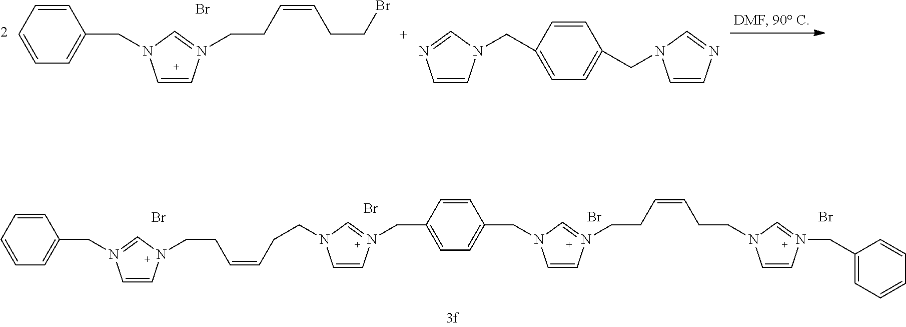

### Sample 3g:

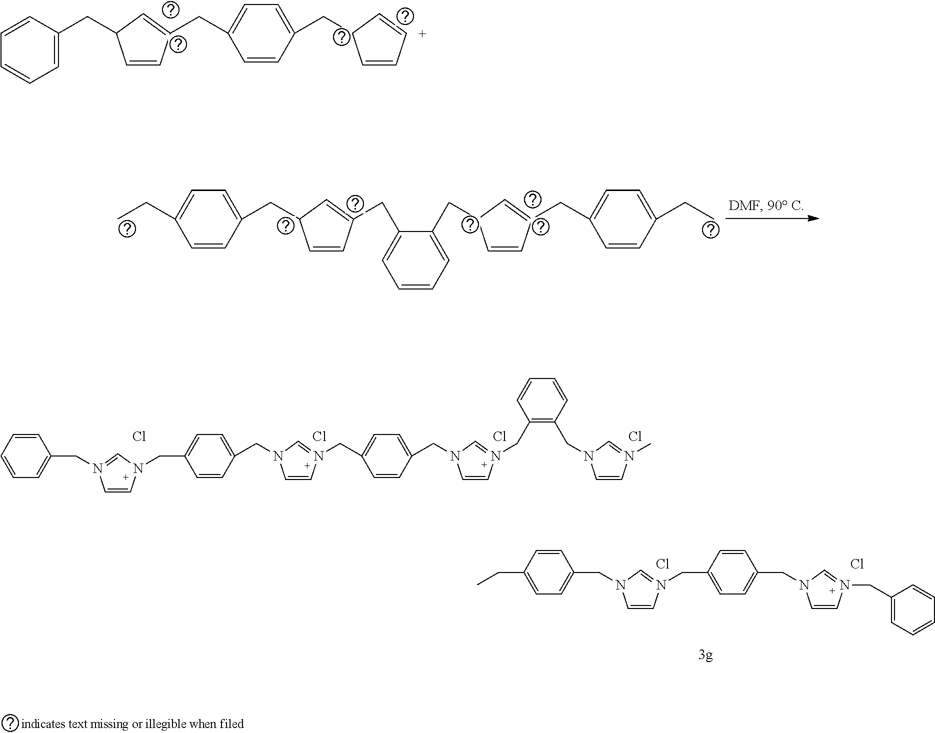

### Sample 3h:

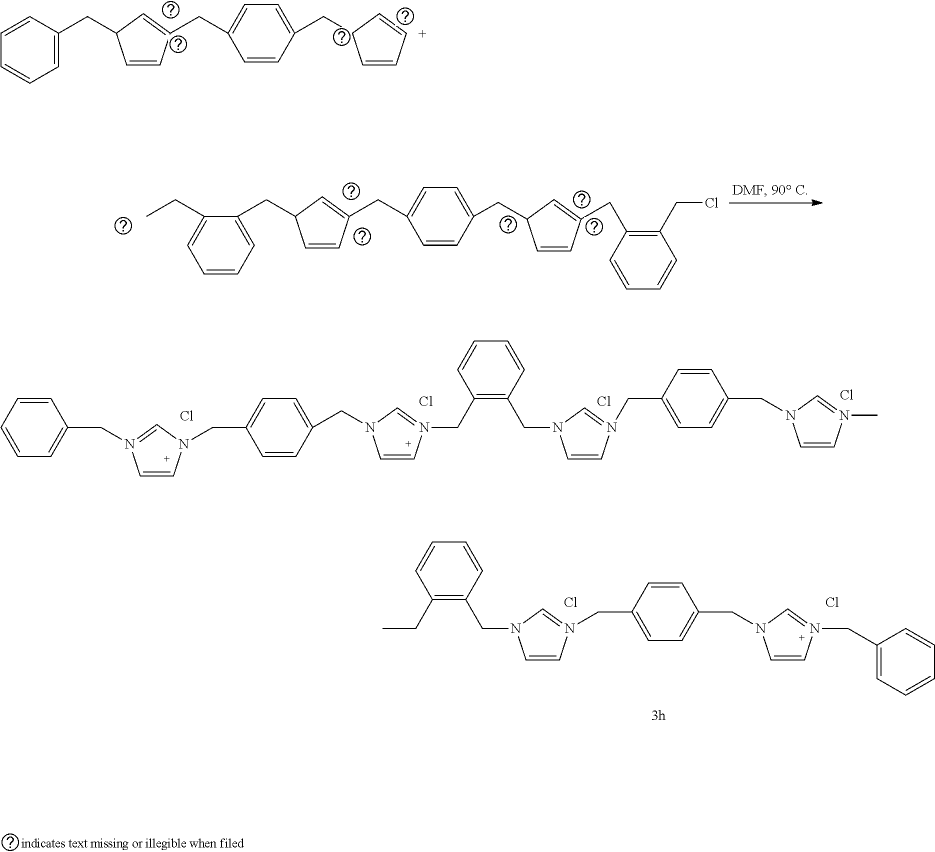

### Example 4

### Antimicrobial and Hemolytic Analysis of Samples 2a to 2t and 3a to 3h

### Protocol for Antimicrobial Analysis:

All bacteria and yeast originated from a −80° C. frozen stock. Bacteria were grown overnight at 37° C. in Tryptic Soy broth (TSB) and yeast was grown overnight at 22° C. in Yeast Mold (YM) broth. Sub-samples of these cultures were grown for three hours further and diluted to an OD600 of 0.1. The bacteria solutions (about 2˜5×10⁸ cells/mL) were then added to the 96-well plate and were incubated at 37° C. for 24 hours. All experiments were run in triplicate and the reported minimum inhibitory concentration (MIC) is the concentration necessary to inhibit complete cell growth.

### Protocol for Hemolytic Activity Analysis:

Fresh rat red blood cells (RBCs) were diluted with phosphate buffered saline (PBS) buffer to give a RBC stock suspension (4 vol. % blood cells). A 100 μL RBC stock solution was added to a 96-well plate containing 100 μL polymer stock solutions (serial 2-fold dilution in PBS). After an hour of incubation at 37° C., the plate was centrifuged at 4000 rpm for 5 min. Hemolytic activity was determined as a function of hemoglobin release by measuring OD₅₇₆ of 100 μL of the supernatant. A control solution that contained only PBS was used as a reference for 0% hemolysis. 100% hemolysis was measured by adding 0.5% Triton-X to the RBCs.

$$Hemolysis ( \% ) = \frac{{OD}_{576 polymer} - {OD}_{576 blank}}{{OD}_{576 Triton - X 100} - {OD}_{576 blank}} \times 100$$

Using the above protocol for antimicrobial analysis, the MIC of each of the 28 samples were determined for the following microorganisms: Bacillus subtilis (ATCC 23857, gram-positive), Escherichia coli (ATCC 25922, gram-negative) and Candida albicans (ATCC 10231, yeast), and the results were tabulated into Table 1 below.

Using the above protocol for hemolytic activity analysis, the polymer concentration necessary for 50% lysis of RBC (HC₅₀^{a}) of 18 samples were determined, and the results were tabulated into Table 2 below.

TABLE 2

| | MIC (μg/ml) | | | HC₅₀ |
|---|---|---|---|---|
| Sample | E. Coli | B. subtilis | C. albicans | μg/ml |
| 2a | >62 | >62 | >62 | >500 |
| 2b | >125 | 31-62.5 | 125 | >500 |
| 2c | >500 | >500 | >500 | — |
| 2d | >500 | >125 | >62 | >500 |
| 2e | >120 | — | >120 | — |
| 2f | 30 | 15 | 80 | >500 |
| 2g | 60 | 30 | 80 | >500 |
| 2h | 30 | 7.8 | 60 | >500 |
| 2i | 30 | 15 | 62.5 | >500 |
| 2j | 40 | 7.8 | 125 | 250 |
| 2k | 40 | 15.6 | 125 | 50 |
| 2l | 20 | 7.8 | 35 | >500 |
| 2m | 60 | 30 | >250 | >500 |
| 2n | 60 | 30 | 125 | — |
| 2o | >500 | 125-250 | 62.5 | — |
| 2p | >125 | 62.5 | 62.5 | >500 |
| 2q | >62 | 15.6 | 32 | >500 |
| 2r | 62.5 | 10 | 35 | >500 |
| 2s | 35 | 7.8 | 35 | >500 |
| 2t | 80 | 40 | 125 | >500 |
| 3a | >250 | >250 | >250 | — |
| 3b | >250 | >250 | >250 | — |
| 3c | >250 | >250 | >250 | — |
| 3d | >250 | >250 | >250 | — |
| 3e | 31 | 31-62 | >250 | — |
| 3f | >250 | >250 | >250 | — |
| 3g | 3.9 | <3.9 | 62.5-125 | >500 |
| 3h | 7.8 | <3.9 | 62.5-125 | >500 |

Comparative examples (Samples 2a, 2b, 2c, 2d, 2e, 2m, 2n, 2o and 2t): From the results in Table 2, it can be seen that there is a significant loss in the antimicrobial property of the polymer when the polymer formed does not have a sufficiently flexible structure either due to bonding of the imidazole to a sp² carbon (samples 2a, 2b, 2c, 2d and 2e), use of overly rigid linkers (biphenyl linker in samples 2m, 2n and 2t), or when ⁻the linker molecule is too small (methyl linker in sample 2o).

With regard to samples 3a to 3h, it is demonstrated that oligomers require a minimum of 6 imidazolium units before showing significant antimicrobial activity. However, it is noted that oligomers having 4 imidazolium units (samples 3e) also display some level of antimicrobial activity against selected strains of bacteria.

Using the above protocol for hemolytic activity analysis, further analysis of 9 polymer and 2 oligomer samples (samples 2f, 2h, 2i, 2j, 2k, 2l, 2q, 2r, 2s, 3g and 3h) was carried out.

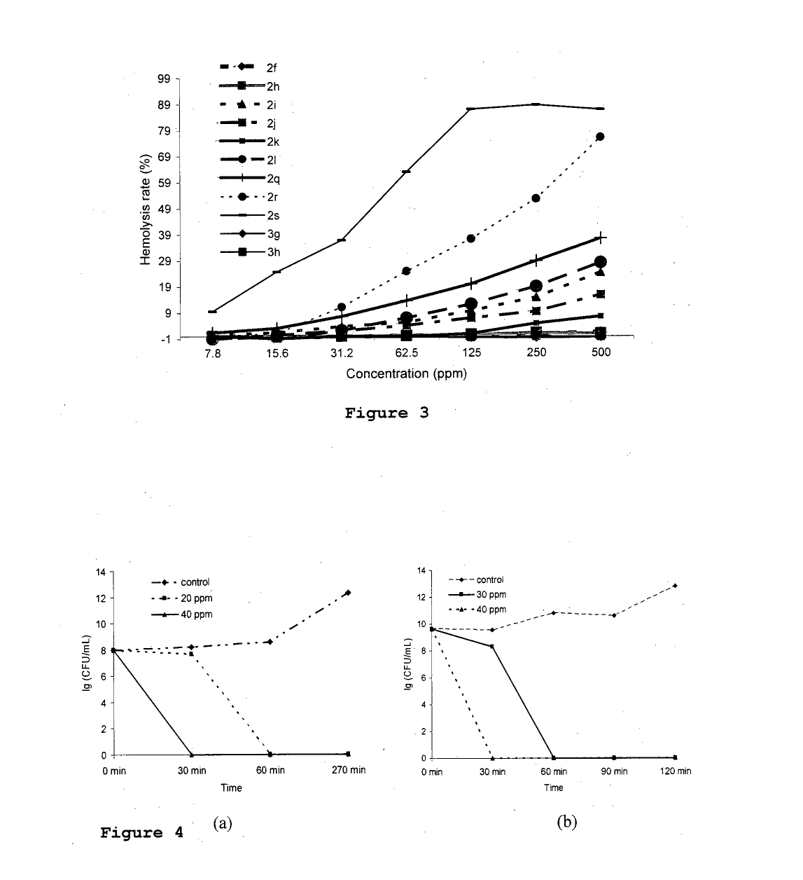

There is shown in FIG. 3, hemolytic activity graphs of active antimicrobial polymer and oligomer samples 2f, 2h, 2i, 2j, 2k, 2l, 2q, 2r, 2s, 3g and 3h. There is shown, significant hemoglobin leakage for samples 2j and 2k, modest hemoglobin leakage for samples 2i, 2q, 2r, 2s and 3h and almost undetectable hemoglobin leakage for samples 2f, 2h, 2l and 3g. This result demonstrates the exemplary antimicrobial and non-hemolytic properties of the samples 2f, 2h, 2l and 3g.

Three of the best performing samples were tested further on antimicrobial activity, in particular using the four microorganisms listed below, which have each shown resistance to current antimicrobial compounds: Vancomycin-resistant enterococcus (isolated from patient, gram-positive), Methicillin-resistant Staphylococcus aureus (isolated from patient, gram-positive), Klebsiella pneumoniae (isolated from patient, gram-negative), and Cryptococcus neoformans (Fluconazole-resistant yeast). The results are as displayed in Table 3 below.

TABLE 3

| | MIC (μg/ml) | | | |
|---|---|---|---|---|
| Sample | VRE | MRSA | K. pneumoniae | C. neoformans |
| 2h | 15 | 7.8 | 7.8 | 7.8 |
| 2l | 31.25 | 6 | 6 | 7.8 |
| 3g | 15.6 | 3 | 3 | 7.8 |

From Table 3, the 3 samples, 2h, 2l and 3g have exhibited the ability to eliminate microorganisms resistant to present antimicrobial treatments at an MIC similar to that of microorganisms without resistance. This indicates that the mechanism of action for the polymer and oligomers synthesized is not the same as that of present antimicrobial compounds.

### Example 5

### Evaluation of the Antimicrobial Activity of Sample 2f as a Function of Time

B. subtilis and E. coli were grown overnight in TSB at 37° C. The grown cells were then diluted to 2˜5×10⁸ CFU/mL and a 100 μL aliquot of this suspension was then added to a sample of TSB broth with no polymer added and samples of TSB broth containing 15, 30 and 40 ppm of polymer sample 2f, respectively.

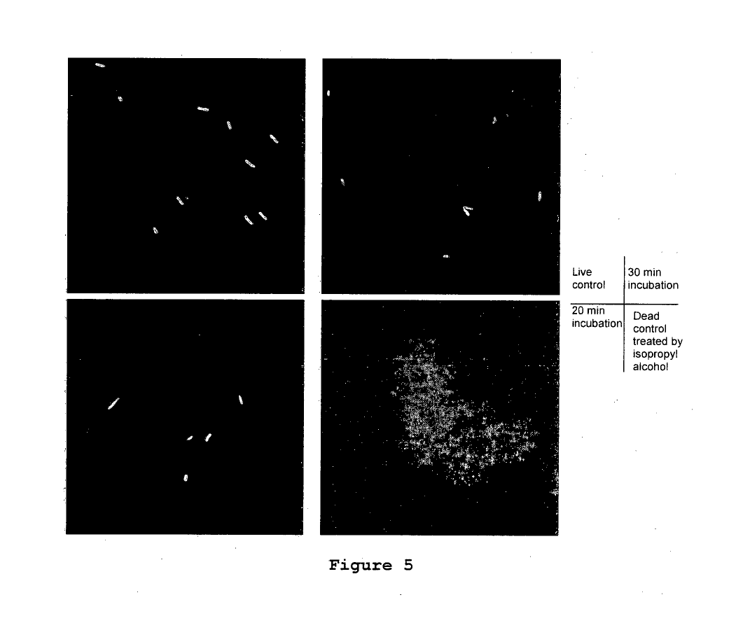

Sample aliquots were withdrawn from all cultures immediately after polymer addition (t=0 h) and also at 0.5, 1, 2, 4 and 6 hour time period after the polymer was added. The aliquots were plated on solid lysogeny broth (LB) agar plates and incubated at 37° C. overnight before colony numbers were counted. The experiments were performed in duplicate and plate counts were averaged, before the resulting values were plotted on a log scale against time. FIGS. 4(a) and 4(b) show the antimicrobial efficiency of sample 2f in colonies of B. subtilis and E. coli respectively. For both types of baterial colonies, sample 2f when used at a concentration of 40 μg/ml, exhibits the ability to lyse all microorganisms present in the broth within the first 30 minutes. Thus the primary bactericidal mechanism of action of the polymer appears to be independent of protein synthesis. In agreement with in vitro killing assay, confocal photographs (FIG. 5) clearly indicated that E. coli bacteria were killed within 30 min.

### Example 6

### Red Blood Cell Agglutination Studies

Agglutination and morphology change of RBCs were investigated using an optical microscope (Olympus IX71)). The 4% RBC suspension was added to the same volume of test compound solution (15.6 to 1000 ppm in serial 2-fold dilutions). After an hour of incubation at 37° C., 20 μL of each sample was diluted with PBS and observed with the microscope. All pictures were taken at a magnification of 400×.

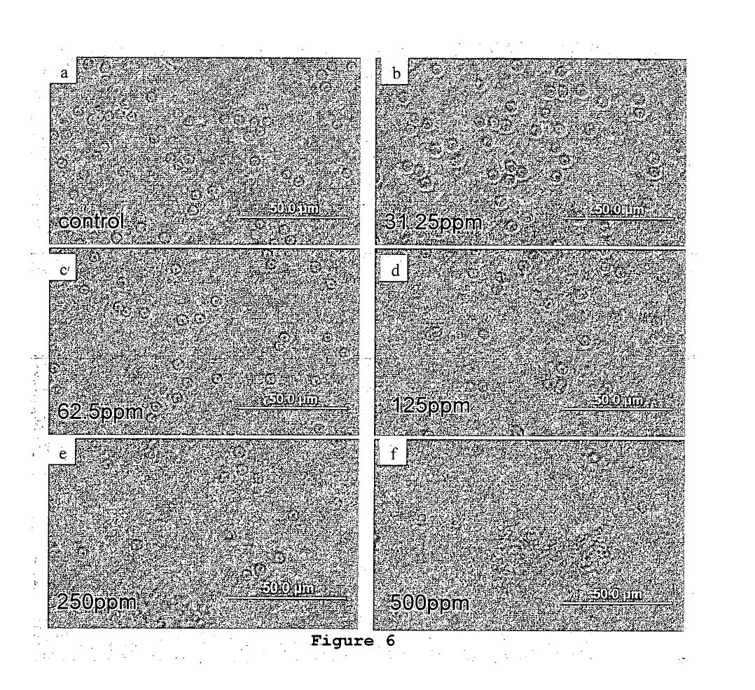

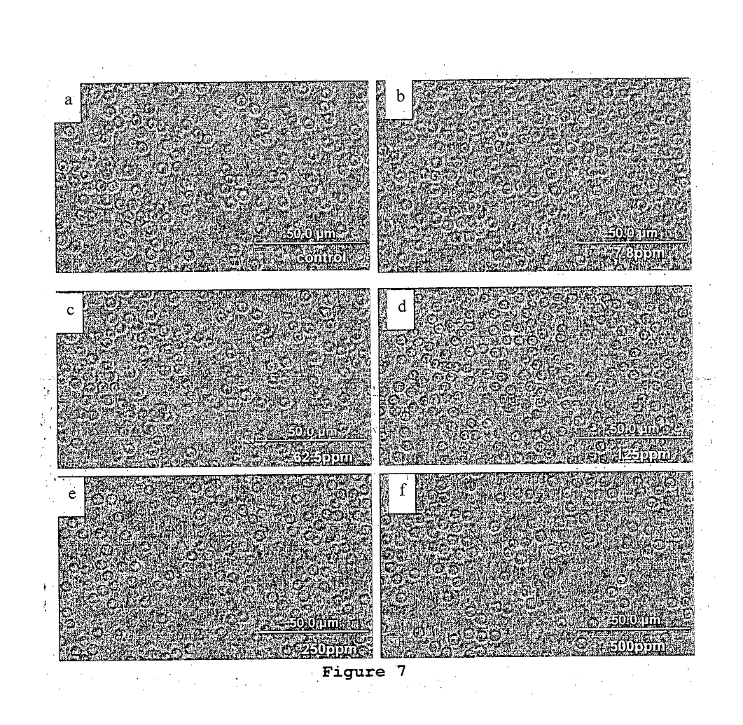

As shown in FIGS. 6(a) to 6(f), further study of red blood cell morphology by light microscopy revealed that sample 2f (and 2h) caused RBC agglutination without any lysis from 62.5 μg/ml (FIG. 6(c)). The agglutinate size increased with higher concentrations of the polymer, up to the maximum concentration of 500 μg/ml investigated. Erythrocytes agglutination often results from ionic interactions, between sialic acid present on RBC membranes and cationic imidazolium groups on the MCPIM and MCOIM. In contrast, polymer 2l and oligomer 3g demonstrated advantageous and unexpected non-erythrocyte fusion and non-erythrocyte agglutination characteristics. As shown in FIGS. 7(a) to 7(h), blood cell morphology was well maintained even when subjected to the maximum concentration of 500 μg/ml of polymer sample 2l.

### Confocal Microscopy of E. Coli.

To visualise bacterial morphology changes after incubation with polymer solutions, E. coli was stained with a mixture of SYTO9 dye and propidium iodide for 15 min at room temperature. A solution containing a sample polymer was then added at minimum inhibition concentration. After 20 and 30 minutes interval, 10 μL of the polymer/bacteria solution was deposited on a microscope slide, covered by a coverslip, and placed into a Carl Zeiss LSM 510 META upright confocal microscope for the images to be taken.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

### Applications

The disclosed antimicrobial composition sees utility in the field of medical treatment and further in the manufacture of commercial products where sterility is desired, such as medical equipment, such as clothing, bed linen, gloves, clean room suits, etc. Advantageously, as the disclosed antimicrobial compositions derive their bioactivity from a relatively mechanical action on the cytoplasmic membrane, it becomes less likely for pathogens to develop resistance against such antimicrobial agents. Furthermore, as the disclosed antimicrobial agents are active against most microorganisms having a cytoplasmic membrane, the disclosed antimicrobial compositions can be envisioned for use as broad spectrum antibiotics.

Additionally, the disclosed antimicrobial compositions have been found to be selectively toxic towards the pathogenic microoganisms, while remaining relatively harmless to other types of somatic cells, such as, red blood cells. Therefore, the disclosed compositions advantageously exhibit a low level of cytotoxicity relative to their lethality towards bacteria.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. An antimicrobial composition comprising at least one polymer or oligomer, said polymer and oligomer being comprised of repeating units of hydrophilic heterocyclic amine monomers that are coupled by hydrophobic linkers selected to confer the antimicrobial activity to the composition.

2. The composition as claimed in claim 1, wherein said heterocyclic amine monomer units are selected from heterocyclic amines having a 4-membered ring, a 5-membered ring or a 6-membered ring.

3. The composition as claimed in claim 2, wherein said heterocylic amine monomers are selected from the group consisting of: azetidines, dihydroazetes, pyrolidine, imidazolidine, triazolidine, tetrazolidine, pentazolidine, pyrrole, imidazole, triazole, pyridine, piperidine, diazinane, triazinane, pyrimidine, triazines and combinations thereof.

4. The composition as claimed in any one of the preceding claims, wherein the hydrophobic linker is selected from an optionally substituted aryl group and an aliphatic olefin.

5. The composition as claimed in any one of the preceding claim, wherein said heterocyclic amine monomer unit is an imidazole.

6. The composition as claimed in any one of the preceding claims, wherein said polymer or oligomer comprises repeating units of general formula (I), 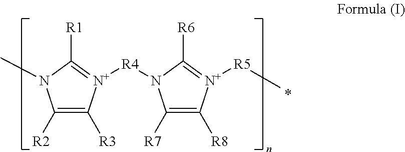
wherein:
R4 and R5 are independently selected from the group consisting of optionally substituted aryl and an aliphatic olefin and;
R1, R2, R3, R6, R7, and R8 are independently selected from hydrogen, alkyl, alkenyl, aryl, halogen and amines; and
n is an integer of at least two.

7. The composition as claimed in claim 6, wherein n is from 2 to 50.

8. The composition as claimed in claim 6, wherein n is from 6 to 8.

9. The composition as claimed in claim 6, wherein n is in the range 4 to 10 for the oligomer.

10. The composition as claimed in claim 6, wherein n is in the range of 8 to 50 for the polymer.

11. The composition as claimed in any one of claims 6 to 10, wherein R4 and R5 are independently selected from the group consisting of: xylene, aliphatic C₂₋₆ alkylene, phenylbenzene, substituted phenylbenzene, and combinations thereof.

12. The composition as claimed in claim 13, wherein R4 and R5 are independently selected from the group consisting of: ortho-xylene, para-xylene, meta-xylene, pyridine, propylene, butylene, pentylene, substituted bi-phenyl, propene, ethene, and combinations thereof.

13. The composition as claimed in claim 12, wherein the bi-phenyl is 1-methyl-4-(4-methylphenyl)benzene.

14. The composition as claimed in any one of claims 6 to 13, wherein R4 is o-xylene and R5 is butylene.

15. The composition as claimed in any one of claims 6 to 14, wherein R1, R2, R3, R6, R7, and R8 are each hydrogen.

16. The composition as claimed in any one of claims 6 to 15, wherein the polymer is provided as a halide salt.

17. The composition of claim 16, wherein said halide is fluoride, bromide, chloride or iodide.

18. The composition as claimed in any one of claims 1 to 17, wherein said oligomer comprises at least four imidazolium units, each imidazolium unit being coupled to an adjacent imidazolium unit via a hydrophobic linker molecule A and said imidazolium unit having the general formula (II) 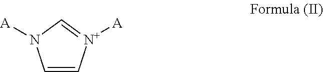
wherein said linker molecule A is independently selected from an optionally substituted aryl and an aliphatic olefin.

19. The composition as claimed in claim 18, wherein said oligomer has at least six imidazolium units.

20. The composition as claimed in claim 18 or claim 19, wherein A is selected from the group consisting of:

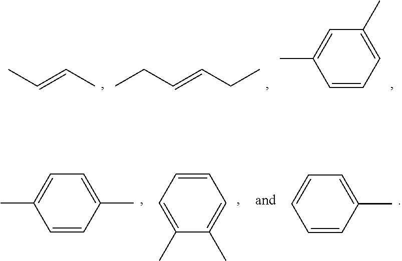

21. An composition according to any one of claims 1 to 20, wherein said oligomer is selected from the group consisting of:

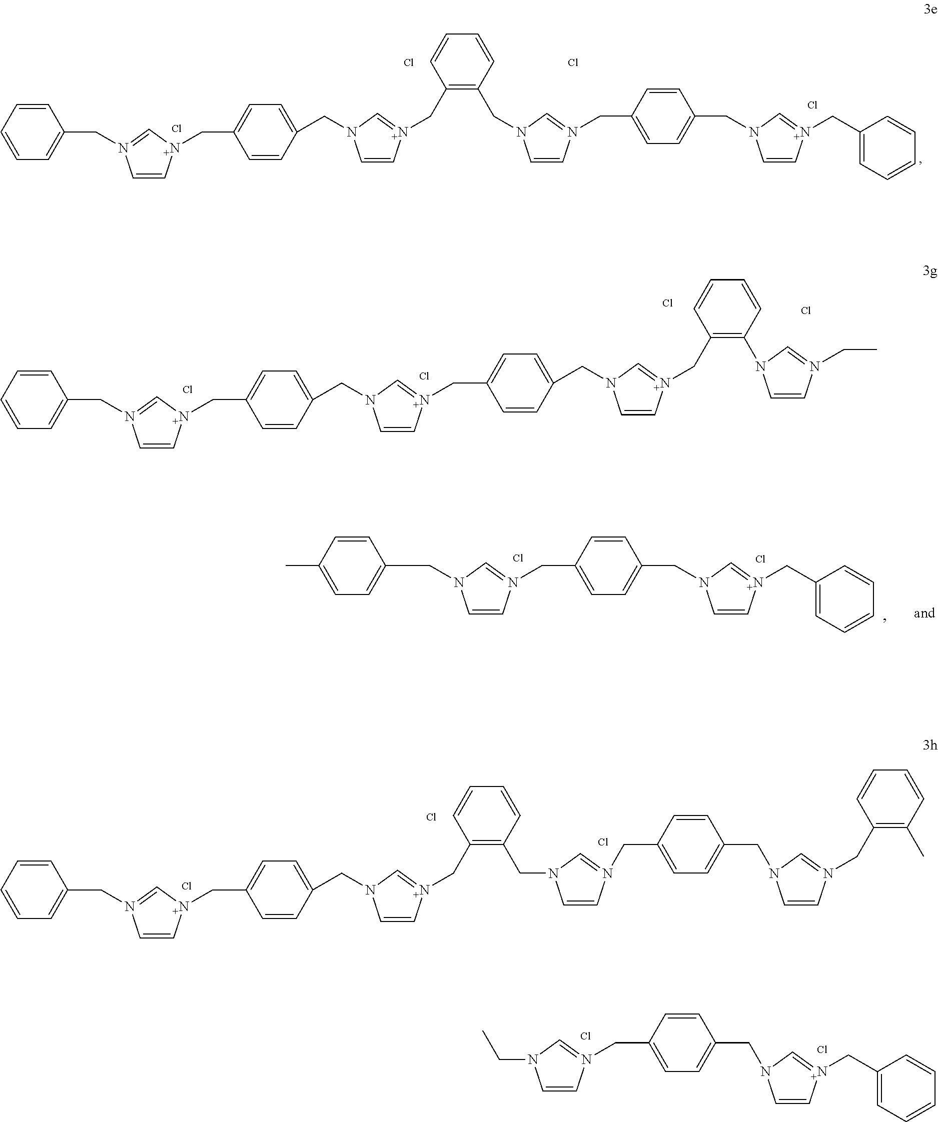

22. The composition as claimed in any one of the preceding claims, wherein said oligomer is provided as an oligomeric halide salt.

23. The composition as claimed in claim 22, wherein the halide of the oligomeric halide salt is selected from fluoride, bromide, chlorine or iodide.

24. A microbial cream comprising the antimicrobial composition as claimed in any one of claims 1 to 23 and one or more pharmaceutically acceptable excipients suitable for topical administration.

25. Use of the composition of any one of claims 1 to 23, for the preparation of a medicament for treating bacterial infections.

26. The use of claim 25, wherein said bacteria causing said bacterial infections is selected from the group consisting of: gram-positive and gram-negative bacteria.

27. The use of claim 26, wherein said bacteria is selected from Bacillus subtilis, Vancomycin-resistant enterococcus, methicillin-resistant Staphylococcus aureus (MRSA), Escherichia coli, Klebsiella pneumoniae, Candida albicans, and Cryptococcus neoformans.

28. Use of the composition according to any one of claim 1 as an antimicrobial agent.

29. A method of producing an amphiphilic polymer or oligomer, said method comprising the step of reacting aryl-substituted heterocyclic amine monomers units with at least one of a heterocyclic amine substituted with one or more haloalkyl arenes, haloalkyl arenes, and a di-halogenated aliphatic olefin, in the presence of an organic solvent, said aryl-substituted heterocyclic amine monomers units having at least two heterocyclic amine groups linked by an aryl group.

30. The method as claimed in claim 29, wherein said method further comprises, prior to said reacting step, a pre-reaction step of reacting an imidazole and di-halogenated xylene to form said aryl-substituted heterocyclic amine monomer unit.

31. The method as claimed in claim 29 or claim 30, further comprising providing said di-halogenated aliphatic olefin from the group comprising of: dibromobutylene, dichlorobutylene, dichloropropene, dibromopropene, dichloroethylene, dibromoethylene, and mixtures thereof.

32. The method as claimed in one of claims 29 to 31, further comprising providing said heterocyclic amine substituted with one or more haloalkyl arenes from the group consisting of:

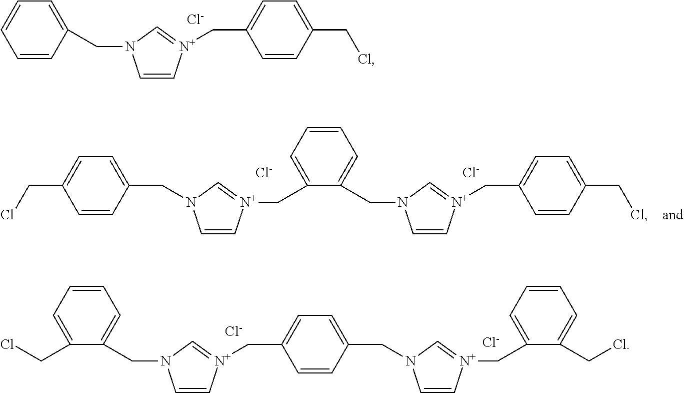

33. The method as claimed in one of claims 29 to 32, further comprising providing said haloalkyl arene from the group consisting of: dibromo-m-xylene, dibromo-p-xylene, dibromo-o-xylene, dichloro-m-xylene, dichloro-p-xylene, dichloro-o-xylene, 4,4′-bis(chloromethyl)-1,1′-biphenyl and mixtures thereof.
